(19) United States
(12) Patent Application Publication
(10) Pub. No.: US 2018/0311456 A1
(43) Pub. Date: Nov. 1, 2018

(54) Filtering Mask with Adaption to Face Shape and Method for Improving the Airtightness of Filtering Mask

(71) Applicant: ZHONGKE SUNEN TECHNOLOGY (BEIJING) CO., LTD, Beijing (CN)

(72) Inventor: Bin TIAN, Beijing (CN)

(21) Appl. No.: 15/693,240

(22) Filed: Aug. 31, 2017

(30) Foreign Application Priority Data
Apr. 28, 2017 (CN) 201710291703.8

(51) Int. Cl. A61M 16/06; A41D 13/11; A62B 23/02; A41H 43/04

(52) U.S. Cl. CPC A61M 16/0622; A41D 13/1138; A62B 23/025; A41D 2500/50; A41D 2500/30; A61M 2205/0216; A41H 43/04

(57) ABSTRACT

A mask includes a body. An edge portion of the body is provided with a sealed member fitted and adapted to a human face, and a space allowing gas or liquid to be filled therein is provided in the sealed member. During wearing and using, since the sealed member filled with gas or liquid is used as a contact structure for the mask contacting with the human face, the sealed member filled with gas or liquid is elastic and can be deformed when contacting with the human face. Thus, in actual wearing, such a gas-filled or liquid-filled structure can enable the sealed member to self-adapt to different facial features due to the deformation, which ensures the airtightness of the mask, thereby ensuring filtering effectiveness of the mask. A method of improving mask airtightness applied to the mask is further provided according to the present application.

### CROSS-REFERENCE TO RELATED APPLICATIONS

The present application claims the benefit of priority to Chinese patent application No. 201710291703.8 titled “FILTERING MASK WITH ADAPTION TO FACE SHAPE AND METHOD FOR IMPROVING THE AIRTIGHTNESS OF FILTERING MASK”, filed with the Chinese State Intellectual Property Office on Apr. 28, 2017, the entire disclosure of which is incorporated herein by reference.

### FIELD

This application relates to the technical field of respiratory filters, and in particular to a mask. The present application further relates to a method of improving mask airtightness applied to the mask.

### BACKGROUND

Masks are becoming a necessity in people's lives. In one aspect, intrusions from substances such as smell, dust and fine particles in life are always unavoidable, in another aspect, air pollution is becoming more and more serious, and haze seriously affects lives of ordinary people. Therefore, anti-haze air filtering masks also become a necessity for people's lives. After a mask is designed, a size of the mask is fixed, however, facial features of people are different, it is inevitable that the mask does not closely fit a human face when a user wears a mask, since the mask may have a different size with the facial feature of the individual. Such not fitting situation mainly presents at a part having large difference, such as wings of a nose, a nasal fossa and a chin. This results in the following major problems. Firstly, for the anti-haze mask, air filtering cotton is provided on a body of the mask, but if an edge of the mask is not fitted closely to the human face and thus air may enter the mask from the edge of the mask without passing through the filtering cotton, the filtering effectiveness of the anti-haze mask is weakened greatly; secondly, for an ordinary mask, the gas exhaled by a human body should avoid discharging from a gap between the wings of a nose and the mask and a gap between the nasal fossa and the mask as much as possible, since this may cause discomfort to eyes in one aspect, and in the another aspect, for a person wearing glasses, the exhaled gas is apt to be condensed on lens, causing poor sight and further causing personal safety risks.

Therefore, an important technical issue to be addressed by the person skilled in the art currently is to improve a fitting degrees and airtightness between the mask and the human face when the mask is worn.

### SUMMARY

A mask, which can closely fit a human face when being worn and has a high airtightness, is provided according to the present application. A method of improving mask airtightness applied to the above mask is provided according to the present application.

A mask is provided according to the present application, which includes a body. An edge portion of the body is provided with a sealed member fitted and adapted to a human face, and a space filled with gas or liquid is provided in the sealed member.

Preferably, an exterior of the sealed member is covered with a wrapping member.

Preferably, the wrapping member is made of cotton fabric, flannelette or non-woven fabric.

Preferably, the sealed member is a member made of a soft elastic material.

Preferably, the sealed member is any one of a silica gel member, a sponge member, a latex member, a polyvinyl chloride member or a TPU (thermoplastic polyurethane) member.

A method of improving mask airtightness applied to the mask according to any one of the above aspects is further provided according to the present application, and the method includes the following steps:

preassembly, including: assembling the sealed member adapted to the edge portion of the body of the mask in a sealed form to form a sufficient sealed space in the sealed member and to allow the sealed member to fit and adapt to a human face; and

substrate assembly, including: connecting the sealed member to the edge portion of the body reliably.

Preferably, in the step of preassembly, gas or liquid is introduced into the space in the sealed member, and an interior of the sealed member may be filled up with gas or may alternatively not be filled up with gas.

Preferably, in the step of substrate assembly, the sealed member and the body are connected by any one of stitching, bonding and ultrasonic welding.

Compared with the above background, during wearing and using of the mask according to the present application, since the sealed member filled with gas or liquid is used as a contact structure for the mask contacting with the human face, the sealed member filled with gas or liquid is elastic and can be deformed when contacting with the human face. Thus, in actual wearing, such a gas-filled or liquid-filled structure can enable the sealed member to self-adapt to different facial features due to the deformation, which ensures that human faces with different facial structures can be fully matched with and closely fitted to the mask and ensures airtightness of the mask at an edge in use, thereby ensuring filtering effectiveness of the mask.

In another preferred embodiment of the present application, an exterior of the sealed member is covered with a wrapping member. The wrapping member is capable of wrapping the sealed member entirely or partially to improve tactility of a user and avoid structural damage due to direct exposure of the sealed member to the outside.

### BRIEF DESCRIPTION OF THE DRAWINGS

For more clearly illustrating embodiments of the present application or the technical solutions in the conventional technology, drawings referred to describe the embodiments or the conventional technology will be briefly described hereinafter. Apparently, the drawings in the following description are only examples of the present application, and for the person skilled in the art, other drawings may be obtained based on the drawings without any creative efforts.

FIG. 1 is a front view showing the structure of a mask according to an embodiment of the present application;

FIG. 2 is a schematic exploded structural view of the mask in FIG. 1;

FIG. 3 is a schematic view showing the structure of a sealed member in FIG. 1; and

FIG. 4 is a flow chart of a method of improving mask airtightness according to an embodiment of the present application.

### DETAILED DESCRIPTION OF EMBODIMENTS

A mask which can be highly fitted to a human face and has a high airtightness when being worn is provided according to the present application. A method of improving mask airtightness applied to the mask is further provided.

In order to make the person skilled in the art have a better understanding of a solution of the present application, the present application is described hereinafter in further detail in conjunction with the drawings and embodiments.

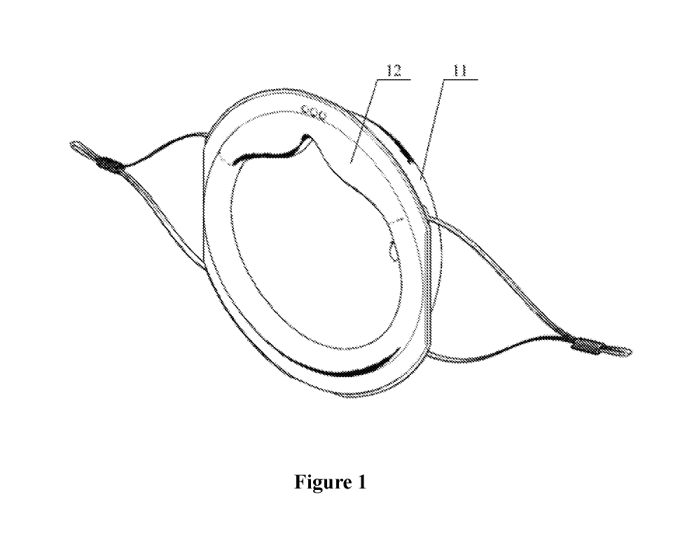

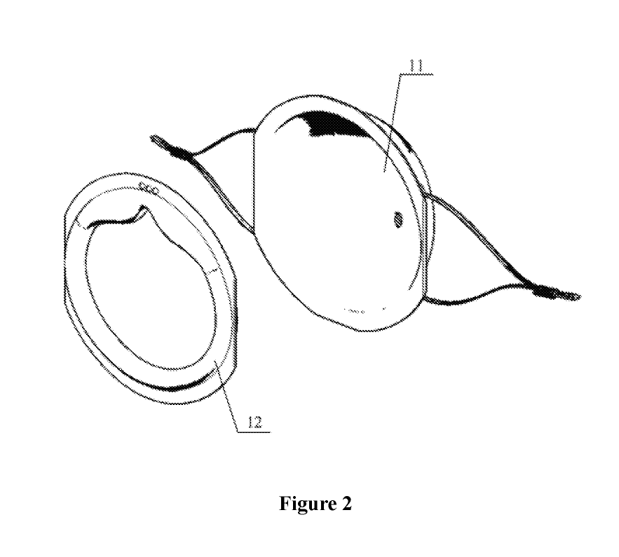

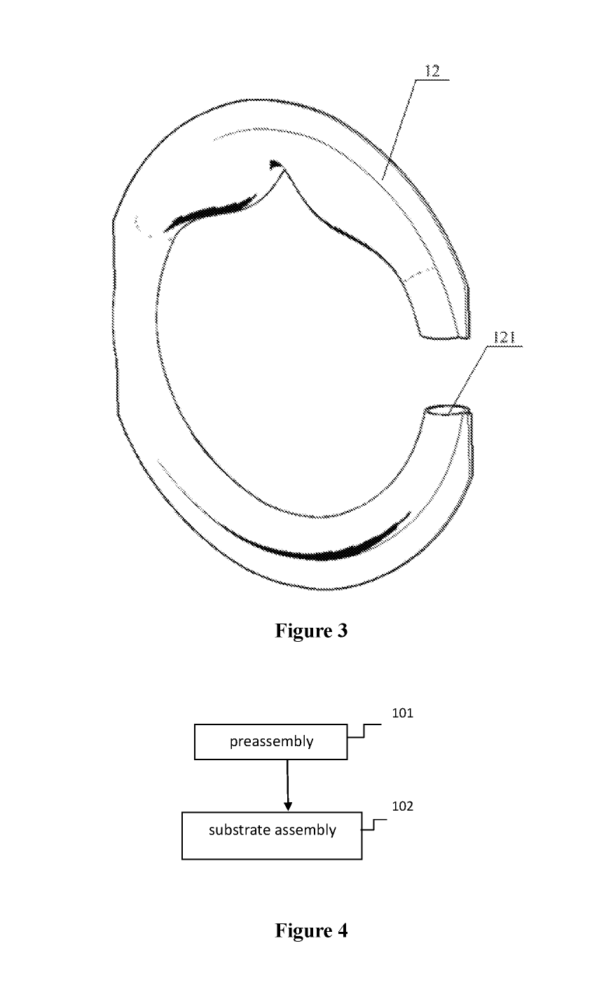

Reference is made to FIGS. 1 and 2. FIG. 1 is a front view showing the structure of a mask according to an embodiment of the present application and FIG. 2 is a schematic exploded structural view of the mask in FIG. 1.

In an embodiment, a mask according to the present application includes a body 11. An edge portion of the body 11 is provided with a sealed member 12 fitted and adapted to a human face. A space 121, allowing gas or liquid to be filled therein, is provided in the sealed member 12.

In a wearing and using process, since the sealed member 12 filled with gas or liquid is employed as a fitting structure for the mask to contact with the human face, the sealed member filled with gas or liquid is elastic and can be deformed when contacting with the human face. Thus, such a gas-filled or liquid-filled structure can allow the sealed member 12 to self-adapt to different facial features in actual wearing due to the deformation, which ensures that human faces with different facial structures can be fully matched with and closely fitted to the mask in wearing and ensures airtightness of the mask at an edge in use, thereby ensuring filtering effectiveness of the mask.

It should be noted that, particularly in a practical application, in the case that gas is introduced into the sealed member 12, the gas in the sealed member 12 typically does not fill up the space 121, so as to ensure the inflated sealed member 12 to still have a variable structure and a high deformation buffer capacity. Of course, according to requirements of an actual working condition, the staff may adjust filling amount and pressure of gas in the sealed member 12 flexibly and choose whether to fill up the sealed member 12 with the gas, so as to meet wearing requirements of different users in different usage environments.

In addition, in a special working condition, the sealed member 12 is only required to be assembled in a sealed form, without requiring introducing gas into an interior of the sealed member, so as to further reduce production costs, simplify production processes, and, meet usage requirements in some extreme working conditions.

Furthermore, an exterior of the sealed member 12 is covered with a wrapping member (not shown in the figure). The wrapping member can wrap the sealed member 12 entirely or partially, which improves the tactility of a user and avoids structural damage due to the direct exposure of the sealed member 12 to the outside.

More specifically, the wrapping member is made of cotton fabric. This cotton fabric has good tactility and a low cost, which can reduce an overall production cost of the mask while ensuring the user to have comfort tactility.

In another aspect, the sealed member 12 is a gasbag. This airbag has a simple and reliable structure, has a high charging efficiency and is easy to be manufactured, and has an inexpensive cost, which can simplify an overall structure of the mask effectively and reduce the manufacturing cost of the mask accordingly.

Also, the sealed member 12 is a member made of soft elastic material. Such a member made of a soft elastic material has a good flexibility and is capable of fitting human faces with different facial features sufficiently, which ensures usage effectiveness of the mask and has a certain enclosing and buffer capacity.

In addition, the sealed member 12 is any one of a silica gel member, a sponge member, a latex member and a polyvinyl chloride member. The silica gel member, the sponge member, the latex member and the polyvinyl chloride member are common members of soft skin-friendly materials, and have a large market supply in raw materials and have low purchasing costs, which can reduce an overall manufacturing cost of the mask accordingly. Of course, selection of materials of the above-mentioned sealed member 12 is not limited to the above, and the sealed member 12 may be a member of any other types of soft elastic skin-friendly materials as long as it can meet production and use requirements of the mask.

Reference is made to FIG. 4, which is a flow chart of a method of improving mask airtightness according to an embodiment of the present application.

In an embodiment, a method of improving mask airtightness according to the present application is applied to the mask as described above, the method includes:

step 101, preassembly; and step 102, substrate assembly.

In step 101, the sealed member 12 adapted to the edge portion of the body 11 of the mask is assembled in a sealed form such that an enough sealed space 121 is formed inside the sealed member 12 so as to allow the sealed member 12 to adapt to and fit a human face.

Further, in the preassembly of step 101 described above, gas or liquid is introduced into the space 121 inside the sealed member 12, however, the sealed member 12 may be filled up with gas and may alternatively not be filled up with gas. The interior of the sealed member 12 may be maintained in a filling up state or not in a filling up state according to actual demands, which ensures the sealed member 12 to have a certain elastic deformation capability, thereby avoiding adversely affecting the fitting effectiveness of the sealed member 12 with the human face and the airtightness of the mask in a condition that the interior of the sealed member 12 is preassembled over full or has an over high gas pressure.

In step 102, the sealed member 12 is connected to the edge portion of the body 11 reliably.

More specifically, in the substrate assembly of step 102 described above, a connection manner for connecting the sealed member 12 to the body 11 is stitching or bonding. These two connection manners are simple to operate and reliable in structure, and can simplify manufacturing processes of the mask and improve reliability of product structures of the mask.

Of course, particularly in a practical application, the connection form for connecting the above sealed member 12 and the body 11 is not limited to the bonding or stitching described above, and the sealed member 12 and the body 11 may also be connected in a stuck manner or a sleeving manner. In the case that special materials such as some engineering plastics are employed, the sealed member 12 and the body 11 may alternatively be connected in a special process such as ultrasonic welding. In principle, any connection manners can be employed as long as the connection manner can meet the requirements of practical operation of the method of improving mask airtightness.

It may be known from above that the mask provided in the present application includes a body, and an edge portion of the body is provided with a sealed member which is fitted and adapted to a human face, and a space, which allows gas or liquid to be filled therein, is provided in the sealed member. In a wearing and using process, since the sealed member filled with gas or liquid is employed as a contact structure for the mask contacting with the human face, the sealed member filled with gas or liquid is elastic and can be deformed when contacting with the human face. Thus, in actual wearing, such a gas-filled or liquid-filled structure can enable the sealed member to self-adapt to different facial features due to the deformation, which ensures that the human faces with different facial structures all can be fully matched with and closely fitted the mask when the users wear the mask and ensures airtightness of the mask at an edge in use, thereby ensuring filtering effectiveness of the mask.

In addition, the method of improving mask airtightness applied to the above-mentioned mask according to the present application is simple and easy to operate, and the mask products thereof can be closely fitted to a human face and has a high airtightness.

The mask according to the present application and the method of improving mask airtightness applied to the mask are described above in detail. Specific examples are applied herein to set forth principles and embodiments of the present application, and description of the above embodiments is merely to help understand the method of the present application and its core ideas. It should be noted that it is apparent to the person skilled in the art that various improvements and modifications may be made to the present application without departing from the principles of the present application, and these improvements and modifications also fall within the scope of claims of the present application.

1. A mask, comprising:
a body,
wherein an edge portion of the body is provided with a sealed member fitted and adapted to a human face, and a space filled with gas or liquid is provided in the sealed member.

2. The mask according to claim 1, wherein an exterior of the sealed member is covered with a wrapping member.

3. The mask according to claim 2, wherein the wrapping member is made of cotton fabric, flannelette or non-woven fabric.

4. The mask according to claim 1, wherein the sealed member is a member made of a soft elastic material.

5. The mask according to claim 4, wherein the sealed member is any one of a silica gel member, a sponge member, a latex member, a polyvinyl chloride member or a thermoplastic polyurethane member.

6. A method of improving the airtightness of a mask, the method comprising steps:
preassembly, comprising: assembling the sealed member adapted to the edge portion of the body of the mask in a sealed form to form a sufficient sealed space in the sealed member and to allow the sealed member to fit and adapt to a human face; and
substrate assembly, comprising: connecting the sealed member to the edge portion of the body reliably.

7. The method of improving mask airtightness according to claim 6, wherein in the step of preassembly, gas or liquid is introduced into the space in the sealed member, and an interior of the sealed member is filled up with gas or is not filled up with gas.

8. The method of improving mask airtightness according to claim 6, wherein in the step of substrate assembly, the sealed member and the body are connected by any one of stitching, bonding and ultrasonic welding.
